(19) United States
(12) Patent Application Publication
(10) Pub. No.: US 2016/0328665 A1
(43) Pub. Date: Nov. 10, 2016

(54) MEDICAL WORKFLOW MANAGEMENT METHOD

(71) Applicant: AGFA HEALTHCARE NV, B-Mortsel (BE)

(72) Inventors: Joost FELIX, Mortsel (BE); Hua LIU, Mortsel (BE); Yuping WANG, Mortsel (BE)

(73) Assignee: AGFA Healthcare NV, Mortsel (BE)

(21) Appl. No.: 15/036,858

(22) PCT Filed: Nov. 25, 2014
(86) PCT No.: PCT/EP2014/075507

§ 371 Date: May 16, 2016

(30) Foreign Application Priority Data
Nov. 28, 2013 (EP) 13194844.0

(51) Int. Cl. G06Q 10/06; G06Q 50/22; H04L 29/06

(52) U.S. Cl. CPC G06Q 10/0633; H04L 63/08; G06Q 50/22

(57) ABSTRACT

A method for workflow management in a medical workflow management system wherein a first user logs in to the workflow management system running a desktop application and next identifies an additional, non logged-in user and defines at least one task of the workflow for this additional user. Selection of an additional user and defined task(s) triggers the workflow management system to associate the defined task(s) of the workflow with the additional user.

### CROSS-REFERENCE TO RELATED APPLICATIONS

This application is a 371 National Stage Application of PCT/EP2014/075507, filed Nov. 25, 2014. This application claims the benefit of European Application No. 13194844.0, filed Nov. 28, 2013, which is incorporated by reference herein in its entirety.

### BACKGROUND OF THE INVENTION

1. Field of the Invention

The present invention is in the field of medical image acquisition. The invention more specifically relates to a method for performing clinical workflow tasks.

2. Description of the Related Art

In a medical picture archiving and communication system (PACS) such as IMPAX Agility of Agfa Healthcare N.V. a number of applications are offered which are called ‘desktops’.

A single desktop brings together all the activities of a user with a typical departmental role.

Providing all information and tools for all of the user's tasks on a single desktop, frees the users from the burdens of switching between windows, losing context, dealing with synchronization issues, and so on.

Agfa's IMPAX Agility system comprises a Diagnostic Desktop, a Technologist's desktop and an Administrator desktop.

The Diagnostic desktop is designed for radiologists and cardiologists and supports workflows or tasks such as study viewing, reporting, order review/protocolling support, etc., all from one coherent desktop.

The Technologist Desktop supports typical technologist workflow or tasks, including image acquisition, study preparation and follow-up, quality control tasks, etc.

In the Administrator Desktop system administrators and departmental coordinators can configure and monitor any RIS (radiology information system)/PACS configuration aspects.

Each of these desktops is integrated in the common task management system, allowing fluent coordination of tasks, both ad-hoc for non-standard situations, and automated for routine workflows.

Technologist related tasks or workflows are often completed by multiple physicians.

For example one technologist, one or more assistants or nurses are involved in one image acquisition task.

In the acquisition room often only one workstation running the technologist desktop is available.

In the current situation one user is logged in and only this user is recorded as involved in the preparation/acquisition/follow-up tasks of a given workflow.

In case a task would be completed by another technologist than the one that is logged in, this person must re-login to complete the task.

So assistants are not registered unless they separately log in.

If not separately logged in, their names will not be recorded as responsible operator for this task.

This is cumbersome, requires additional actions from the users and may have negative consequences e.g. for workload statistics.

US patent application 2009/094529 discloses a system for execution of a clinical workflow. The system also allows multiple users to have access to a task list via an interface from different workstations such that different users may execute different actions within a single workflow.

The described features are common for distributed systems involving different workstations but do not provide a solution for the above-described problem.

### SUMMARY OF THE INVENTION

It is thus an aspect of the present invention to provide a method and a system for facilitating completion of clinical workflow or clinical workflow tasks in case of multiple users want to co-work on a single workstation simultaneously.

The above-mentioned aspects are achieved by a method and a system as set out below.

Specific features for preferred embodiments of the invention are also set out below.

Further advantages and preferred embodiments of the present invention will become apparent from the following description and drawings.

### BRIEF DESCRIPTION OF THE DRAWINGS

FIG. 1 is a flow chart illustrating a method of the present invention.

FIG. 2 is a login window in which a logged in person can add a person.

FIG. 3 illustrates the selection of responsible/involved technologist for a certain task.

FIG. 4 schematically shows the components of an apparatus for performing a method of the present invention.

### DETAILED DESCRIPTION OF THE PREFERRED EMBODIMENTS

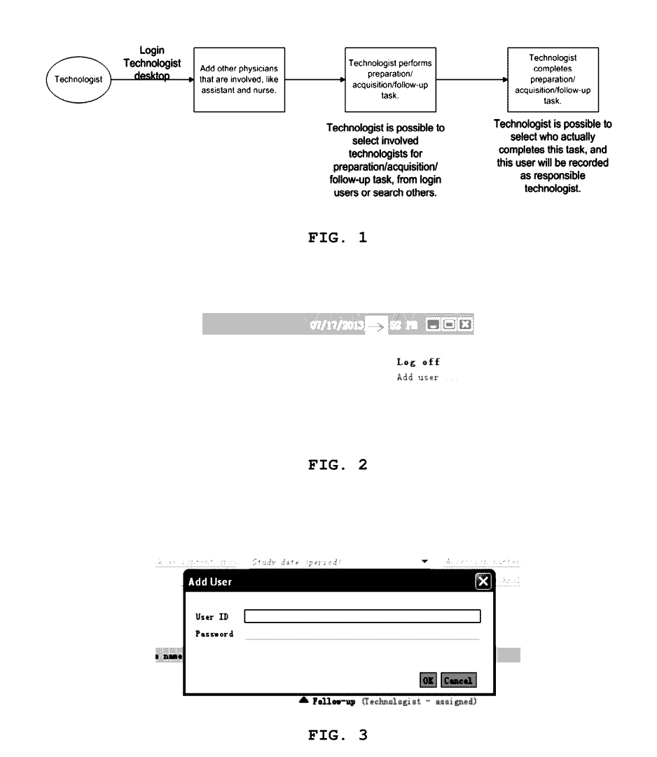

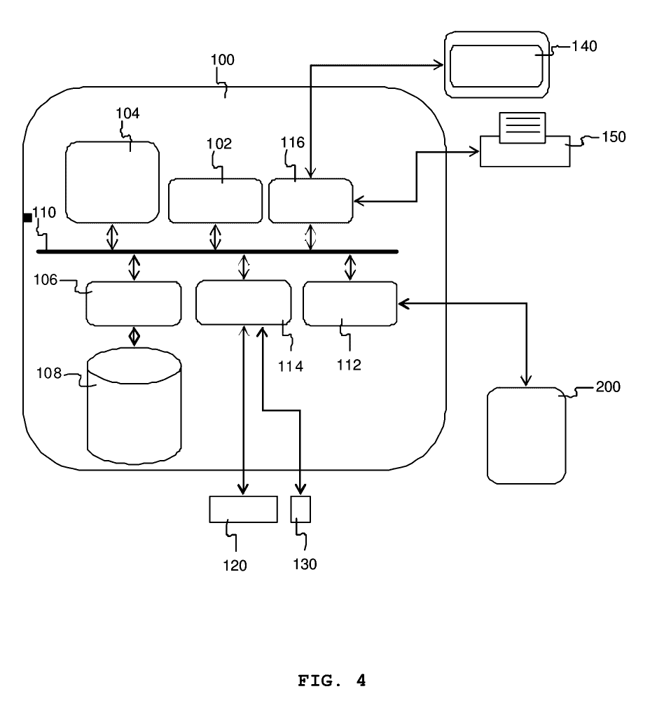

FIG. 1 is a flowchart illustrating the different steps of a preferred embodiment of a method of the present invention.

The invention is explained hereafter with regard to a technologist's desktop but is likewise applicable to any other kind of application in which more than one person is involved. Examples in the field of medical imaging are other desktops such as administrator's desktop or diagnostic desktop.

In a first step a technologist logs in to the technologist's desktop on a login screen. The desktop application provides the possibility to add an additional non logged in user(s) (FIG. 2).

In one preferred embodiment, additional non logged in users may be added by typing in the identification and password for another user known to the picture archiving and communication system.

In another preferred embodiment the available additional non logged in users have been identified in advance. Their identities are stored in the system and can be retrieved and displayed in the technologist's desktop, e.g. in the form of a list with selectable items. In this way, a name of an additional non logged in user can be selected in the displayed list by the logged in user.

Upon addition of additional users or selection of additional users from the list by the primary logged in user, these additional users will have access to the workflow running on the technologist's desktop or to one or more tasks of such a workflow.

For a task (for example a preparation task of image acquisition task or an acquisition task itself or a follow-up task of an image acquisition task) another user, more specifically the user who actually performed and completed the task, can be selected and associated with the task as responsible user while the other, logged in users are recorded as involved users.

So this mode of operation allows several users to work on the same user interface. They will be able to perform different steps of a workflow and mark completion of a task with their own identity from a single available user interface.

In the prior art the logged in user would have been recorded as performer of a task running on a desktop application. Another user could not be selected in a single workstation as performer of a task without having to perform a re-login.

A method of the present invention allows a user to be associated with a task and to be registered as performer of a task without having to perform re-login.

A method of the present invention can be performed in a system as described below.

FIG. 4 shows a suitable computing system 100 for hosting data processing system 1 for workflow management according to a preferred embodiment of the present invention.

Computing system 100 may in general be formed as a suitable general purpose computer, such as a workstation, a server, a laptop, a desktop, a hand-held device, a mobile device, a tablet computer, or other computing device, as would be understood by those of skill in the art.

The computing system 100 comprises a bus 110, a processor 102, a local memory 104, one or more optional input interfaces 114, one or more optional output interfaces 116, a communication interface 112, a storage element interface 106 and one or more storage elements 108.

Bus 110 may comprise one or more conductors that permit communication among the components of the computing system.

Processor 102 may include any type of conventional processor or microprocessor that interprets and executes programming instructions.

Local memory 104 may include a random access memory (RAM) or another type of dynamic storage device that stores information and instructions for execution by processor 102 and/or a read only memory (ROM) or another type of static storage device that stores static information and instructions for use by processor 104.

Input interface 114 may comprise one or more conventional mechanisms that permit an operator to input information to the computing device 100, such as a keyboard 120, a mouse 130, a pen, voice recognition and/or biometric mechanisms, etc.

Output interface 116 may comprise one or more conventional mechanisms that output information to the operator, such as a display 140, a printer 150, a speaker, etc. Communication interface 112 may comprise any transceiver-like mechanism such as for example two 1 Gb Ethernet interfaces that enables computing system 100 to communicate with other devices and/or systems, for example mechanisms for communicating with one or more other computing systems 200.

The communication interface 112 of computing system 100 may be connected to such another computing system by means of a local area network (LAN) or a wide area network (WAN), such as for example the internet, in which case the other computing system 200 may for example comprise a suitable web server.

Storage element interface 106 may comprise a storage interface such as for example a Serial Advanced Technology Attachment (SATA) interface or a Small Computer System Interface (SCSI) for connecting bus 110 to one or more storage elements 108, such as one or more local disks, for example 1 TB SATA disk drives, and control the reading and writing of data to and/or from these storage elements 108. Although the storage elements 108 above is described as a local disk, in general any other suitable computer-readable media such as a removable magnetic disk, optical storage media such as a CD or DVD disk, solid state drives, flash memory, Random Access Memory (RAM), Read Only Memory (ROM), Electronically Erasable Programmable Read Only Memory (EEPROM) or other memory technologies, holographic media, magnetic cassettes, magnetic tape, magnetic disk storage or other magnetic storage devices that can be used to encode information and can be accessed by the computing system 100 could be used.

The processor of the above described computer system 100 runs a desktop application such as a technologist desktop. The technologist's desktop comprises pre-defined workflows comprising a number of workflow steps or tasks to be performed and to be indicated with status ‘completed’ when they have been performed and are completely finished.

The processor is further configured to associate tasks in a clinical workflow with users.

When a user is logged into the desktop he/she is associated with workflows/tasks running on the desktop unless another user is identified and associated with a task or an entire workflow in accordance with the present invention.

A user interface is further provided having a means to identify at least one non logged in user in addition to the logged in user and having means to select a workflow or one or more tasks of a workflow.

The processor is configured to connect at least one of the additionally identified non logged in users to at least one task of said desktop application.

The processor may be further configured to record said additional user as responsible user for said task.

The workflow management system 1 can be implemented as programming instructions stored in local memory 104 of the computing system 100 for execution by its processor 102. Alternatively the workflow management system 1 could be stored on the storage element 108 or be accessible from another computing system 200 through the communication interface 112. Such a plurality of suitably connected computing systems could for example form a system or platform with client-server architecture in which the medical images, reports and tasks are maintained centrally or distributed on one or more servers.

Although the present invention has been illustrated by reference to specific preferred embodiments, it will be apparent to those skilled in the art that the invention is not limited to the details of the foregoing illustrative preferred embodiments, and that the present invention may be embodied with various changes and modifications without departing from the scope thereof.

The present preferred embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein. In other words, it is contemplated to cover any and all modifications, variations or equivalents that fall within the scope of the basic underlying principles and whose essential attributes are claimed in this patent application.

It will furthermore be understood by the reader of this patent application that the words “comprising” or “comprise” do not exclude other elements or steps, that the words “a” or “an” do not exclude a plurality, and that a single element, such as a computer system, a processor, or another integrated unit may fulfil the functions of several means recited in the claims.

Any reference signs in the claims shall not be construed as limiting the respective claims concerned. The terms “first”, “second”, third”, “a”, “b”, “c”, and the like, when used in the description or in the claims are introduced to distinguish between similar elements or steps and are not necessarily describing a sequential or chronological order. Similarly, the terms “top”, “bottom”, “over”, “under”, and the like are introduced for descriptive purposes and not necessarily to denote relative positions.

It is to be understood that the terms so used are interchangeable under appropriate circumstances and preferred embodiments of the invention are capable of operating according to the present invention in other sequences, or in orientations different from the one(s) described or illustrated above.

1-5. (canceled)

6. A computer-implemented method for workflow management in a medical workflow management system, the method comprising the steps of:
assigning actions with regard to tasks of a workflow to a user logged-in to the system;
identifying an additional non logged-in user and defining at least one task of the workflow for the additional non logged-in user; and
selecting the additional non logged-in user and the defined at least one task to cause the workflow management system to associate the defined at least one task with the additional non logged-in user.

7. The method according to claim 6, wherein all tasks of the workflow are defined.

8. The method according to claim 6, wherein the step of selecting the additional non logged-in user includes selecting the additional non logged-in user from a predefined number of candidate non logged-in users, wherein identification data of the candidate non logged-in users are displayed on a user interface of the workflow management system.

9. The method according to claim 6, wherein the step of selecting the additional non logged-in user includes selecting the additional non logged-in user by entering a candidate's identification data into a user interface of the workflow management system.

10. A workflow management system comprising:
a processor configured or programmed to associate tasks of a workflow with a logged-in user;
a user interface that displays tasks of the workflow to the logged-in user, the user interface further including means for identifying an additional non logged-in user and for selecting at least one task of the workflow; wherein
selection of the additional non logged-in user and the at least one task causes the processor to associate the selected at least one task with the identified additional non logged-in user.
